(19) United States
(12) Patent Application Publication
(10) Pub. No.: US 2023/0063158 A1
(43) Pub. Date: Mar. 2, 2023

(54) SLICEABLE CONNECTOR FOR LEAD-IN-LEAD CONCEPT

(71) Applicant: Medtronic, Inc., Minneapolis, MN (US)

(72) Inventors: Estelle Fraysse, Eindhoven (NL); Patrick Kleijnen, Mheer (NL); Victor Duijsens, Grevenbicht (NL)

(21) Appl. No.: 17/861,902

(22) Filed: Jul. 11, 2022

Related U.S. Application Data
(60) Provisional application No. 63/237,851, filed on Aug. 27, 2021.

(51) Int. Cl. A61N 1/05; A61N 1/08

(52) U.S. Cl. CPC A61N 1/0563; A61N 1/08

(57) ABSTRACT

An implantable medical lead includes a connector, outer lead, inner lead, electrical contact, and conductive element. The connector is configured to electrically communicate with processing circuitry of a medical device. The inner lead, positioned within a lumen defined by the outer lead, is configured to translate relative to the outer lead. The inner lead includes an inner lead electrode and an inner conductor electrically connected to the inner lead electrode, where the inner conductor is configured to electrically communicate with the connector. The outer lead includes an outer lead electrode and an outer conductor electrically connected to the outer lead electrode. The outer conductor is configured to electrically communicate with the connector.

### RELATED APPLICATIONS

The present application claims the benefit of U.S. Provisional Application No. 63/237,851, filed Aug. 27, 2021, which is incorporated herein by reference in its entirety.

### TECHNICAL FIELD

This disclosure is related to implantable medical systems.

### BACKGROUND

Various types of implantable medical leads have been implanted for treating or monitoring one or more conditions of a patient. Such implantable medical leads may be adapted to allow medical devices to monitor or treat conditions or functions relating to heart, muscle, nerve, brain, stomach endocrine organs or other organs and their related functions. Implantable medical leads include electrodes and/or other elements for physiological sensing and/or therapy delivery. Implantable medical leads allow the sensing/therapy elements to be positioned at one or more target locations for those functions, while the medical devices electrically coupled to those elements via the leads are at different locations.

Implantable medical leads, e.g., distal portions of elongate (e.g., long and slender, but not necessarily stretched) implantable medical leads, may be implanted at target locations selected to detect a physiological condition of the patient and/or deliver one or more therapies. For example, implantable medical leads may be delivered to locations within an atria or ventricle to sense intrinsic cardiac signals and deliver pacing or antitachyarrhythmia shock therapy from a medical device coupled to the lead. In other examples, implantable medical leads may be tunneled to locations adjacent a spinal cord or other nerves for delivering pain therapy from a medical device coupled to the lead. Implantable medical leads may include anchoring components to secure a distal end of the lead at the target location.

### SUMMARY

In an example, an implantable medical lead comprises: a connector configured to electrically communicate with processing circuitry of a medical device; an outer lead comprising: an outer lead electrode; and an outer conductor electrically connected to the outer lead electrode; an inner lead positioned within a lumen defined by the outer lead, wherein the inner lead is configured to translate within the lumen relative to the outer lead, and wherein the inner lead comprises: an inner lead electrode; and an inner conductor electrically connected to the connector, wherein the inner conductor is configured to establish electrical communication between the inner lead electrode and the connector, and wherein the connector is configured to establish electrical communication between the inner lead electrode and the processing circuitry using the inner conductor; an electrical contact electrically connected to the outer conductor; and a conductive element positioned within the lumen, wherein the conductive element is electrically connected to the connector, wherein the conductive element is configured to establish electrical communication between the connector and the electrical contact as the inner lead translates relative to the outer lead, and wherein the connector is configured to establish electrical communication between the outer lead electrode and the processing circuitry using the outer conductor when the conductive element establishes electrical communication between the connector and the electrical contact.

In an example, a method comprises: translating an inner lead of an implantable medical lead comprising a connector configured to electrically communicate with processing circuitry of a medical device, wherein the inner lead is positioned within a lumen defined by an outer lead of the implantable medical lead, and wherein the outer lead comprises: an outer lead electrode; and an outer conductor electrically connected to the outer lead electrode; and wherein the inner lead comprises: an inner lead electrode; and an inner conductor electrically connected to the connector, wherein the inner conductor is configured to establish electrical communication between the inner lead electrode and the connector, and wherein the connector is configured to establish electrical communication between the inner lead electrode and the processing circuitry using the inner conductor; and establishing electrical communication between the outer conductor and the connector, wherein an electrical contact and a conductive element maintain the electrical communication between the outer conductor and the connector as the inner lead translates relative to the outer lead, wherein the electrical contact is electrically connected to the outer conductor, wherein the conductive element is positioned within the lumen defined by the outer lead, wherein the conductive element is electrically connected to the connector, wherein the conductive element is configured to establish electrical communication between the connector and the electrical contact as the inner lead translates relative to the outer lead, and wherein the connector is configured to establish electrical communication between the outer lead electrode and the processing circuitry using the outer conductor when the conductive element establishes electrical communication between the connector and the electrical contact.

In an example, a system comprises: an implantable medical device; and an implantable medical lead comprising: a connector configured to electrically communicate with processing circuitry of the implantable medical device; an outer lead comprising: an outer lead electrode; and an outer conductor electrically connected to the outer lead electrode; an inner lead positioned within a lumen defined by the outer lead, wherein the inner lead is configured to translate within the lumen relative to the outer lead, and wherein the inner lead comprises: an inner lead electrode; and an inner conductor electrically connected to the connector, wherein the inner conductor is configured to establish electrical communication between the inner lead electrode and the connector, and wherein the connector is configured to establish electrical communication between the inner lead electrode and the processing circuitry using the inner conductor; an electrical contact electrically connected to the outer conductor; and a conductive element positioned within the lumen, wherein the conductive element is electrically connected to the connector, wherein the conductive element is configured to establish electrical communication between the connector and the electrical contact as the inner lead translates relative to the outer lead, and wherein the connector is configured to establish electrical communication between the outer lead electrode and the processing circuitry using the outer conductor when the conductive element establishes electrical communication between the connector and the electrical contact.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example medical device system with an implantable medical lead positioned within a heart.

FIG. 2A is a conceptual diagram of an example implantable medical lead.

FIG. 2B is another conceptual diagram of an example implantable medical lead.

FIG. 3 is a conceptual diagram illustrating an example implantable medical lead including a connector, an outer lead, and an inner lead.

FIG. 4A is a conceptual diagram illustrating an example configuration of an electrical contact, conductive element, and inner lead of an implantable medical lead.

FIG. 4B is a conceptual diagram illustrating another example configuration of an electrical contact, conductive element, and inner lead of an implantable medical lead.

FIG. 5 is a block diagram illustrating circuitry of an example implantable medical device.

FIG. 6 is a flow diagram of an example technique for operating a medical device system.

### DETAILED DESCRIPTION

This disclosure describes an implantable medical lead configured to enable multi-chamber functionality for physiological sensing and/or therapy delivery in the heart of a patient. The implantable medical lead is configured to establish electrical communication between an outer lead and a connector and an inner lead and the connector. The connector is configured to electrically communicate with processing circuitry of a medical device. In examples, the implantable medical lead may be configured to penetrate the septum of a patient's heart in order to deliver electrical stimulations to activate a left bundle branch (LBB), other conduction system tissues, and/or or other ventricular tissues of the heart.

The inner lead is configured to translate relative to the outer lead. In examples, the inner lead is configured to receive a distal force that causes the inner lead to translate relative to the outer lead. In such examples, the inner lead may be configured to penetrate tissue as a clinician causes the inner lead to translate relative to the outer lead. In examples, a clinician may control the translation of the inner lead relative to the outer lead to, for example, control a depth to which the inner lead penetrates the tissue when a fixation mechanism of the implantable medical lead secures the outer lead to the target site. This may allow the inner lead to be substantially positioned at a predetermined location based on, for example, pace mapping. In examples, a clinician may control the translation of the inner lead relative to the outer lead to conduct pace mapping. In some examples, the inner lead may translate relative to the outer lead when the inner lead rotates relative to the inner lead.

The implantable medical lead includes the connector, the outer lead, and the inner lead, where the inner lead is positioned within a lumen defined by the outer lead. The implantable medical lead houses electrical conductors electrically connected to one or more electrodes of the implantable medical lead. For example, the outer lead includes an outer lead electrode and an outer conductor electrically connected to the outer lead electrode. The inner lead includes an inner lead electrode and an inner conductor electrically connected to the inner lead electrode. The implantable medical lead may include additional electrodes on the inner lead and/or the outer lead to which the inner conductor and/or the outer conductor may be connected, respectively.

The inner conductor may be configured to electrically communicate with the outer conductor. In examples, the inner conductor includes conductive fillers that are electrically connected to the connector and other electrical components of the implantable medical lead. For instance, a first conductive filler may establish electrical communication between the connector and the inner lead electrode, and a second conductive filler may establish electrical communication between the connector and an electrical contact and conductive element that are electrically connected to the outer conductor. The conductive fillers may be formed such that the conductive fillers are not in electrical contact with each other. For example, the conductive fillers may be formed as coils that are spaced apart such that no two conductive fillers are in electrical contact with each other. Additionally or alternatively, the conductive fillers may be electrically insulated (e.g., coated with a nonconductive substance, such as a polymer) to prevent electrical communication between each other and allow for independent programming.

The implantable medical lead is configured such that the connector may electrically communicate with both the outer lead electrode of the outer lead and the inner lead electrode of the inner lead as the inner lead is translated (e.g., by a clinician) relative to the outer lead. The connector may be configured to electrically communicate with the outer lead electrode and the inner lead electrode via the inner conductor, as discussed in greater detail below. In examples, the connector is configured such that electrical communication between the connector and the outer lead electrode may occur substantially independently of the electrical communication between the connector and the inner lead electrode. In examples, the connector is configured to remain substantially stationary with respect to the outer lead as the inner lead translates relative to the outer lead.

The implantable medical lead is configured such that the inner conductor is electrically connected to the connector. In examples, the connector includes at least a first conductor and a second conductor (i.e., in some cases, the connector includes more than two conductors, such as four conductors). The connector may be configured to electrically isolate each of the conductors; for example, the first conductor may be electrically isolated from the second conductor. In cases where the inner conductor includes conductive fillers, the first conductor may be electrically connected to a first conductive filler of the inner conductor, and the second conductor may be electrically connected to a second conductive filler of the inner conductor. The implantable medical lead may be configured to establish electric communication between the second conductive filler and the outer conductor. In examples, the second conductive filler electrically communicates with the outer conductor via an electrical contact and a conductive element. For instance, the second conductive filler may be electrically connected to the electrical contact, the electrical contact may be electrically connected to the conductive element, and the conductive element may be electrically connected to the outer conductor.

In some examples, the electrical contact is configured to translate relative to the outer lead when the inner lead translates relative to the outer lead. In some examples, the electrical contact is substantially secured to the inner lead such that translation of the inner lead relative to the outer lead causes the translation of the electrical contact relative to the outer lead. The electrical contact may be configured to substantially maintain electrical communication between the outer conductor and the connector (e.g., via the conductive element) as the inner lead causes the translation of the electrical contact.

The electrical contact may be configured to establish and/or substantially maintain the electrical communication between the connector and the outer conductor as the inner lead translates relative to the outer lead, such that the connector may electrically communicate with both the inner conductor and the outer conductor as the inner lead translates relative to the outer lead. In examples, the electrical contact may be positioned within the lumen of the outer lead. In some examples, the electrical contact is elongate (e.g., long and slender, but not necessarily stretched) and configured to be flexible. For example, the electrical contact may be formed as a tube defining a passage within which the inner lead is positioned.

The conductive element is configured to establish electrical communication between the outer conductor and the electrical contact as the inner lead translates relative to the outer lead. The conductive element may be positioned within the lumen defined by the outer lead and substantially between the inner lead and the outer lead. In some examples, the outer lead mechanically supports the conductive element, and the conductive element remains stationary with respect to the outer lead when the inner lead translates relative to the outer lead. In other examples, the inner lead mechanically supports the conductive element, and the conductive element translates relative to the outer lead when the inner lead translates relative to the outer lead. In some cases, the conductive element is a coiled spring formed as a ring that is configured to surround the inner lead and mechanically communicate with at least a portion of an outer perimeter of the inner lead and with at least a portion of an inner perimeter of the outer lead.

Thus, an implantable medical lead may be configured such that a conductive pathway energizing the inner lead electrode includes, for example, the inner lead electrode, the inner conductor (e.g., the first conductive filler of the inner conductor), and the first conductor of the connector. The implantable medical lead may be further configured such that a conductive pathway energizing the outer lead electrode includes, for example, the outer lead electrode, the outer conductor, the conductive element, the electrical contact, the inner conductor (e.g., the second conductive filler of the inner conductor), and the second conductor of the connector.

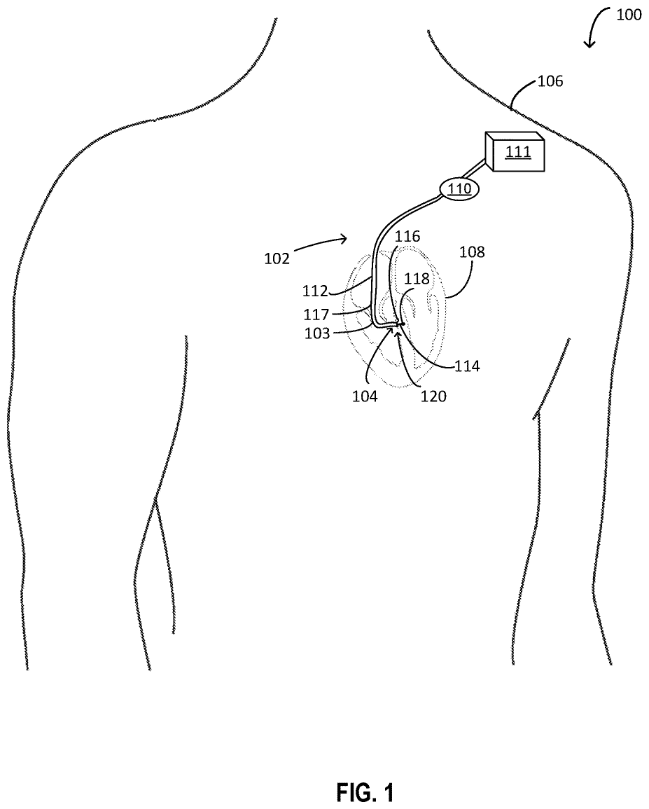

FIG. 1 is a conceptual diagram illustrating a portion of an example medical device system 100 including an implantable medical lead 102. In examples, medical device system 100 is a defibrillator or pacemaker system routinely implanted in patients for the detection and control of tachycardia and/or bradycardia. Implantable medical lead 102 may be configured to sense and/or deliver therapy in a heart 108 of a patient 106. Implantable medical lead 102 may include one or more electrodes for providing electrical therapy to and/or sensing therapy to heart 108. In some examples, implantable medical lead 102 is a quadripolar lead including four electrodes located at a distal portion 103 of implantable medical lead 102.

Implantable medical lead 102 defines a distal portion 103. Distal portion 103 of implantable medical lead 102 may be positioned at a target site 104 within patient 106. In some examples, as illustrated in FIG. 1, the target site 104 may include a portion of heart 108, such as an interventricular septal wall of a right ventricle (RV) of heart 108, as illustrated in FIG. 1, atrioventricular septal wall of a right atrium (RA) of heart 108, or other locations within a body of patient 106. A clinician may maneuver implantable medical lead 102 through the vasculature of patient 106 in order to position distal portion 103 of implantable medical lead 102 at or near target site 104. For example, the clinician may guide distal portion 103 through the superior vena cava (SVC), into the RA, and past the Tricuspid valve into the RV in order to access target site 104 on the atrioventricular septal wall. In some examples, other pathways or techniques may be used to guide distal portion 103 into other target implant sites within the body of patient 106. In some examples, medical device system 100 may include a delivery catheter (not shown), and implantable medical lead 102 may be guided and/or maneuvered within a lumen of the delivery catheter in order to approach target site 104. Distal portion 103 of implantable medical lead 102 may include a distal portion of an outer lead 112 and/or an inner lead 114 of implantable medical lead 102.

Implantable medical lead 102 may include a connector 110 (e.g., a quadripolar connector or IS-4 type connector) configured to electrically communicate with processing circuitry of a medical device 111. Connector 110 may be mechanically supported by implantable medical lead 102. Medical device 111 may be an implantable medical device, such as a defibrillator or a pacemaker. Implantable medical lead 102 includes outer lead 112 and inner lead 114. Outer lead 112 and inner lead 114 may be configured to electrically connect to a single connector (e.g., connector 110). In examples, inner lead 114 may be configured to translate (e.g., slide) relative to outer lead 112 while maintaining electrical communication with connector 110. For example, inner lead 114 may be stationary relative to connector 110, and inner lead 114 may translate relative to outer lead 112 while maintaining electrical communication with connector 110.

Connector 110 may be configured to establish electrical communication between the processing circuitry and both outer lead 112 and inner lead 114. Connector 110 is configured such that the processing circuitry can electrically communicate with outer lead 112 and inner lead 114 substantially independently. For example, the processing circuitry may transmit or sense a first signal via outer lead 112 and transmit or sense a second signal via inner lead 114. In examples, connector 110 includes at least a first conductor configured to establish electrical communication between an electrode of inner lead 114 and the processing circuitry, and a second conductor configured to establish electrical communication between an electrode of outer lead 112 and the processing circuitry. In examples, connector 110 may include additional conductors (e.g., a third conductor, a fourth conductor, etc.) configured to establish electrical communication between other electrodes of inner lead 114 and inner lead 114. In examples, connector 110 includes a connector body mechanically supporting the first conductor and the second conductor. The connector body may be configured to maintain the first conductor and the second conductor substantially stationary relative to each other. The connector body may be configured to electrically insulate at least some portion of the first conductor from the second conductor, and vice-versa. Connector 110 may be, for example, an IS-4 connector or other type of connector including a plurality of conductors mechanically supported by the connector body.

Outer lead 112 of implantable medical lead 102 may include a fixation mechanism 120 configured to grasp tissues at or near target site 104 and substantially secure a distal end of outer lead 112 to target site 104. Fixation mechanism 120 may include, for example, one or more fixation elements. Fixation mechanism 120 may be configured to substantially maintain an orientation of implantable medical lead 102 with respect to target site 104 by penetrating tissues of heart 108. Fixation mechanism 120 may include one or more fixation tines of any shape, including, but not limited to, helically shaped fixation tines. Fixation mechanism 120 may mechanically support or constitute an outer lead electrode 116 such that at least some portion of fixation mechanism 120 is electrically active. For example, fixation mechanism 120 may be an electrode-helix.

Inner lead 114 is positioned within a lumen defined by outer lead 112. Inner lead 114 is configured to translate relative to outer lead 112. For example, inner lead 114 may translate relative to outer lead 112 to penetrate tissues at or in the vicinity of target site 104 when fixation mechanism 120 secures outer lead 112 to target site 104. In examples, inner lead 114 may be configured to receive a force that causes inner lead 114 to translate relative to outer lead 112. In some examples, inner lead 114 is slidably translatable within the lumen defined by outer lead 112. Inner lead 114 may be configured to penetrate tissue as a clinician causes inner lead 114 to translate relative to outer lead 112. Inner lead 114 and/or outer lead 112 may be configured such that a clinician may control the translation of inner lead 114 relative to outer lead 112 to, for example, control a depth to which inner lead 114 penetrates the tissue when fixation mechanism 120 secures outer lead 112 to target site 104. This may allow inner lead 114 to be substantially positioned at a predetermined location based on, for example, pace mapping. In some examples, inner lead 114 may translate relative to outer lead 112 when inner lead 114 rotates relative to outer lead 112.

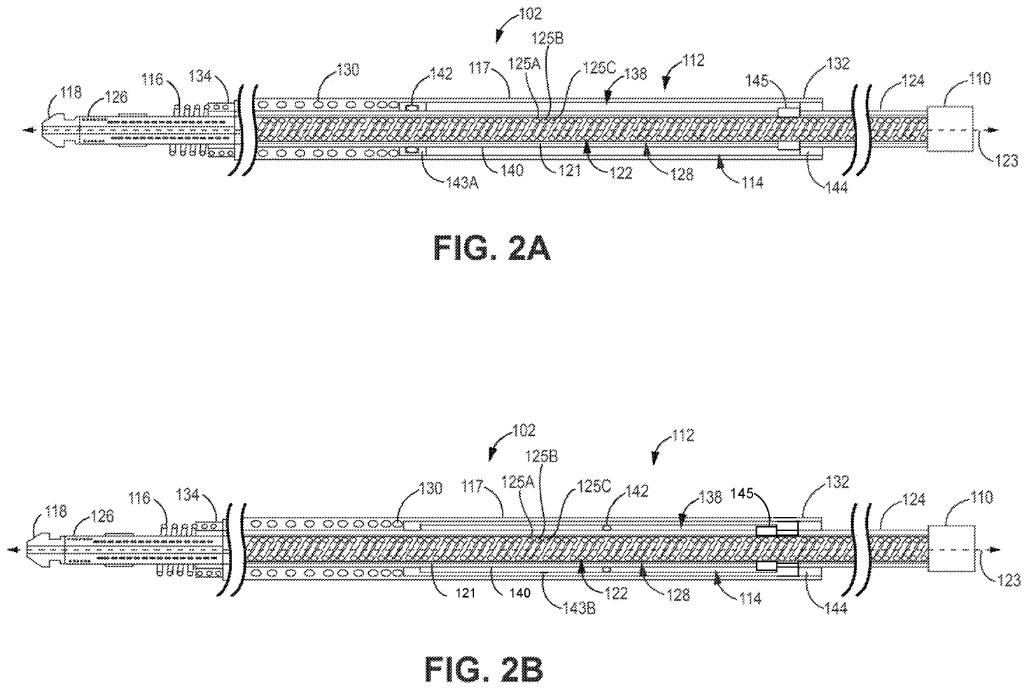

Implantable medical lead 102 houses electrical conductors that are electrically connected to one or more electrodes of implantable medical lead 102. For example, outer lead 112 includes outer lead electrode 116 and an outer conductor (FIGS. 2A and 2B) electrically connected to outer lead electrode 116. The outer conductor may define a helical coil surrounding inner lead 114, although other shapes (e.g., a wire, a tube, etc.) of outer conductor are contemplated by this disclosure. In some examples, outer lead 112 includes an outer lead body 117 mechanically supporting the outer conductor. Outer lead body 117 may define the lumen within which inner lead 114 is positioned. In some examples, the outer conductor is substantially embedded in outer lead body 117. In examples, the outer conductor is configured to electrically connect to a conductor of connector 110 (e.g., the second conductor of connector 110), such that processing circuitry of medical device 111 may electrically communicate with outer lead electrode 116.

Inner lead 114 includes inner lead electrode 118 and an inner conductor (FIGS. 2A and 2B) electrically connected to inner lead electrode 118. Inner lead 114 may support one or more electrodes in addition to inner lead electrode 118. For example, inner lead 114 may support one or more electrodes (e.g., one electrode, two electrodes, three electrodes, etc.), including inner lead electrode 118, positioned near distal end of inner lead 114. The electrodes of inner lead 114 (e.g., inner lead electrode 118) may be configured to pace the LBB or other regions of a heart. As noted above, the inner conductor may be electrically connected to a conductor of connector 110 (e.g., the first conductor of connector 110), such that processing circuitry of medical device 111 may electrically communicate with inner lead electrode 118.

Hence, implantable medical lead 102 may be configured to enable multi-chamber functionality by establishing electrical communication between outer lead electrode 116 and the processing circuitry of medical device 111 (e.g., via the second conductor of connector 110) and establishing electrical communication between inner lead electrode 118 and the processing circuitry of medical device 111 (e.g., via the first conductor of connector 110). Implantable medical lead is configured to substantially maintain the electrical communications as inner lead 114 translates relative to outer lead 112. Connector 110 may be a single, unified connector including a unified body mechanically supporting the first conductor and the second conductor, with implantable medical lead 102 mechanically supporting the unified body. In examples, connector 110 is mechanically supported such that connector 110 is substantially stationery with respect to inner lead 114 as inner lead 114 translates relative to outer lead 112. Thus, implantable medical lead 102 may be configured such that outer lead electrode 116 and inner lead electrode 118 may enable multi-chamber functionality for delivery and/or sensing of signals to or from heart 108 using a single connector such as connector 110 having a unified body, with the single connector configured to remain substantially stationery with respect to inner lead 114 as inner lead 114 translates relative to outer lead 112.

FIGS. 2A and 2B are conceptual diagrams of implantable medical lead 102 of medical device system 100. As shown in FIGS. 2A and 2B, a cross-sectional view of a portion of implantable medical lead 102 is illustrated. Outer lead 112 may define an outer lead axis 123, and inner lead 114 may be configured to translate substantially parallel to outer lead axis 123.

Inner lead 114 may translate relative to outer lead 112 while maintaining electrical communication with connector 110, enabling the positioning and activation of electrodes in multiple areas of a heart (e.g., the right ventricular septum, the left ventricular septum, etc.) using only one implantable medical lead (e.g., implantable medical lead 102) and one connector (e.g., connector 110). Advantages of such a configuration include, for example, reducing the number of surgical tools required to pace/sense several locations (e.g., dual bundle pacing), avoiding the introduction of foreign material into the body of a patient, reducing standard lead implant complications, reducing migration/conduction issues with the implantable medical lead, achieving a patient-specific insertion depth of the implantable medical lead due to the adjustable translation of an inner lead, etc. One or more of these advantages may simplify implantation of medical device system 100 and improve patient outcomes.

As shown in FIGS. 2A and 2B, an inner conductor 122 is operably coupled to (e.g., mechanically supported by) inner lead 114. For example, inner conductor 122 may be disposed within an inner lead body 121 configured to protect inner conductor 122. Inner lead 114 may surround inner conductor 122. For example, inner lead body 121 may define a lumen 128 configured to receive inner conductor 122. Inner conductor 122 and lumen 128 may extend from a proximal end 124 of inner lead 114 (“inner lead proximal end 124”) to a distal end 126 of inner lead 114 (“inner lead distal end 126”). Inner lead body 121 may be formed from an electrically insulative material to electrically isolate at least a portion of inner conductor 122.

Inner conductor 122 is electrically connected to connector 110. Inner conductor 122 is also electrically connected to inner lead electrode 118, which in examples is disposed proximate to inner lead distal end 126. Inner conductor 122 may be a conductive coil (e.g., inner conductor 122 may define a helical coil) formed from a suitable material, such as a metal or an alloy thereof (e.g., MP35N alloy), although inner conductor 122 may be any shape and formed from any material permitting electrical communication between electrodes supported by inner lead 114 and connector 110.

In examples, inner conductor 122 includes conductive fillers (e.g., conductive pathways, such as a conductive coil, conductive traces, conductive wires, etc.), such as conductive fillers 125A-125D (collectively, “conductive fillers 125”), configured to establish electrical communication between electrodes (e.g., outer lead electrode 116, inner lead electrode 118, etc.) of implantable medical lead 102 and connector 110. As shown in FIGS. 2A and 2B, conductive fillers 125 may be formed in the shape of a conductive coil forming various conductive pathways between conductors of connector 110 and electrodes of implantable medical lead 102. For example, conductive filler 125A may be electrically connected to a first conductor of connector 110 and inner lead electrode 118, and conductive filler 125B may be electrically connected to a second conductor of connector 110 and outer lead electrode 116, as discussed in greater detail below. Conductive fillers 125C and 125D may likewise be electrically connected to respective conductors of connector 110 and respective electrodes of implantable medical lead 102 (e.g., other electrodes supported by inner lead 114). Conductive fillers 125 may be formed as coils that are spaced apart such that no two conductive fillers of conductive fillers 125 are in electrical contact with each other. Additionally or alternatively, conductive fillers 125 may be electrically insulated (e.g., coated with a nonconductive substance, such as a polymer) to prevent electrical communication between conductive fillers 125 and allow for independent programming.

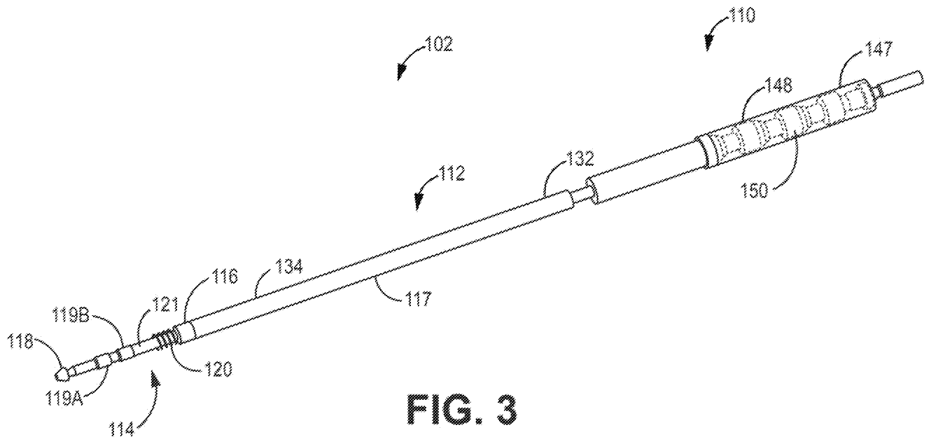

An outer conductor 130 is operably coupled to (e.g., mechanically supported by) outer lead body 117. For example, outer conductor 130 may be embedded in outer lead body 117. In some examples, outer lead body 117 may define a lumen 138 extending from a proximal end 132 of outer lead 112 (“outer lead proximal end 132”) to a distal end 134 of outer lead 112 (“outer lead distal end 134”). A length of outer lead body 117 may be less than a length of inner lead body 121 such that when outer lead distal end 134 and inner lead distal end 126 are aligned, inner lead proximal end 124 is exposed (as shown in FIG. 3). This difference in lengths may permit translation of inner lead 114 relative to outer lead 112.

Inner lead body 121 may be positioned within lumen 138. In such examples, outer conductor 130 may surround at least a portion of inner lead 114. Outer conductor 130 may extend from outer lead proximal end 132 to outer lead distal end 134 or a portion thereof. Outer conductor 130 is electrically connected to outer lead electrode 116, which in examples is disposed proximate to outer lead distal end 134. Outer conductor 130 may be a conductive coil formed from a suitable material, such as a metal or an alloy thereof (e.g., MP35N alloy), although outer conductor 130 may be any shape formed from any material permitting electrical communication between the one or more electrodes supported by outer lead 112 and connector 110.

Implantable medical lead 102 may be configured to establish electric communication between outer conductor 130 (including the electrodes supported by outer lead 112) and connector 110 via an electrical contact 140 and a conductive element 142. Electrical contact 140, which may also be referred to as a long electrode, may be mechanically supported by a portion of inner lead body 121; for example, electrical contact 140 may be positioned within lumen 138 and surround inner lead body 121. As shown in FIGS. 2A and 2B, the diameter of the portion of inner lead body 121 mechanically supporting electrical contact 140 may be different from the diameter of other portions of inner lead body 121. For example, the diameter of the portion of inner lead body 121 mechanically supporting electrical contact 140 may be less than the diameter of other portions of inner lead body 121 such that the combined diameter of the portion of inner lead body 121 mechanically supporting electrical contact 140 and of electrical contact 140 is substantially equal (e.g., within about 20%, within about 10%, within about 5%, and/or within about 1%) to the diameter of other portions of inner lead body 121.

Electrical contact 140 is configured to electrically communicate with connector 110 while allowing translation of inner lead 114. In cases where inner lead body 121 is disposed between inner conductor 122 and electrical contact 140, one or more of conductive fillers 125, such as conductive filler 125B, may extend transversely through a portion of inner lead body 121 to electrically connect (e.g., abut) to electrical contact 140.

Electrical contact 140 may be configured to translate relative to outer lead 112 when inner lead 114 translates relative to outer lead 112. In this case, conductive filler 125B may be in abutment with but not secured to electrical contact 140. Electrical contact 140 may be configured to remain substantially stationary relative to inner lead 114. In this case, conductive filler 125 may be secured to electrical contact 140. In some examples, electrical contact 140 may be formed as a tube defining a passage within which inner lead body 121 is positioned. In general, electrical contact 140 may be electrically insulated from inner conductor 122 by inner lead body 121. Electrical contact 140 may include a platinum-iridium alloy, a conductive polymer, and/or the like.

Conductive element 142 is configured to establish electrical communication between outer conductor 130 and electrical contact 140 as inner lead 114 translate relative to outer lead 112, in this way establishing electrical communication between the one or more electrodes of outer lead 112 and connector 110. Conductive element 142 may be electrically connected (e.g., in physical contact) to electrical contact. In examples, conductive element 142 remains stationary with respect to outer lead 112 when inner lead 114 translates relative to outer lead 112. In other examples, conductive element 142 translates relative to outer lead 112 when inner lead 114 translates relative to outer lead 112. As shown in FIG. 2A, conductive element 142 may be operably coupled to a support component 143A. Support component 143A may be configured to house at least a portion of conductive element 142 while allowing conductive element 142 to electrically connect to inner conductor 122. For example, support component 143A may be conductive and electrically connected to both conductive element 142 and outer conductor 130. In the example of FIG. 2A, conductive element 142 is secured to support component 143A. Thus, outer lead body 117 may mechanically support conductive element 142 such that conductive element 142 is secured to outer lead body 117. For example, support component 143A, in which conductive element 142 may be positioned, may be attached to outer lead body 117.

Alternatively, as shown in FIG. 2B, inner lead body 121 may mechanically support conductive element 142 such that conductive element 142 remains substantially stationary with respect inner lead body 121 or at least translates relative to outer lead 112. In these cases, conductive element 142 may be operably coupled to a support component 143B. Support component 143B may be elongate and define a lumen in which conductive element 142 may translate. Conductive element 142 may be a coiled spring formed as a ring that is configured to surround electrical contact 140, and thus inner lead body 121, and mechanically and electrically communicate with at least a portion of an outer perimeter of electrical contact 140 and with at least a portion of an inner perimeter of support component 143B. Support component 143B, which may encapsulate conductive element 142, may be attached to outer lead body 117.

Implantable medical lead 102 may include a proximal seal 144 proximate to outer lead proximal end 132 and disposed within lumen 138. Proximal seal 144 may be configured to prevent fluid, such as blood, from migrating into outer lead 112 through outer lead proximal end 132. A transition ring 145 may be configured to control translation of inner lead 114 relative to outer lead 112. For example, with respect of the example of FIG. 2A, when inner lead is being translated in a proximal direction relative to outer lead 112, translation ring 145 may contact proximal seal 144, thereby preventing further translation of inner lead 114 in the proximal direction. Similarly, with respect to the example of FIG. 2B, when inner lead 114 is being translated in a proximal direction relative to outer lead 112, transition ring 145 may contact support component 143B, thereby preventing further translation of inner lead 114 in the proximal direction. In any case, transition ring 145 may surround (e.g., secured to an outer perimeter of) a proximal portion of electrical contact 140 and positioned distal to proximal seal 144. Transition ring 145 may be a buffer, protrusion, stopper, or other feature.

FIG. 3 is a conceptual diagram illustrating implantable medical lead 102 electrically connected to connector 110. Connector 110 may be configured to deliver electrical stimulation from a therapy delivery circuitry of medical device 111, which in examples is a pacemaker or defibrillator, to implantable medical lead 102. Additionally, or alternatively, connector 110 may be configured to deliver electrical signals from heart 108 sensed by implantable medical lead 102 to sensing circuitry of medical device 111. In examples where medical device 111 is an implantable cardioverter defibrillator (ICD), implantable medical lead 102 may be configured to deliver electric shocks to heart 108 in response to an event such as a heart attack or ventricular tachycardia. In some examples, medical device 111 may provide both pacing and defibrillation capabilities, and may perform biventricular or other multi-site resynchronization therapies, such as cardiac resynchronization therapy (CRT).

As shown in FIG. 3, connector 110 includes a plurality of conductors, such as a first conductor 148 and a second conductor 150. First conductor 148 may be electrically isolated from second conductor 150. In examples, connector 110 includes connector body 147 mechanically supporting first conductor 148 and second conductor 150. It should be understood, however, that connector 110 may include additional conductors, such as a third conductor and a fourth conductor, the descriptions of which may be substantially similar to the descriptions of first conductor 148 and second conductor 150. Connector body 147 may be configured to electrically insulate at least some portion of first conductor 148 from second conductor 150, and vice-versa. Connector body 147 may be configured to maintain first conductor 148 and second conductor 150 substantially stationary relative to each other.

Connector 110 is configured to energize, via conductors (e.g., first conductor 148, second conductor 150, etc.), electrodes (e.g., outer lead electrode 116 of outer lead 112, inner lead electrode 118 of inner lead 114, etc.) of implantable medical lead 102 by delivering electrical stimulation from therapy delivery circuitry of medical device 111. It should be understood that connector 110 may be configured to energize electrodes other than outer lead electrode 116 and inner lead electrode 118 (e.g., using conductors other than first conductor 148 and second conductor 150) supported by implantable medical lead 102, including, but not limited to, electrode 119A and electrode 119B, both of which are mechanically supported by inner lead distal end 126 in the example of FIG. 3. First conductor 148 may be configured to electrically connect with one of conductive fillers 125 of inner conductor 122, such as conductive filler 125A. Second conductor 150 may be configured to electrically connect with another one of conductive fillers 125 of inner conductor 12, such as conductive filler 125B. In examples, first conductor 148 is mechanically coupled to conductive filler 125A, and second conductor 150 is mechanically coupled to conductive filler 125B. First conductor 148 and second conductor 150 may be mechanically coupled to conductive fillers 125 using a solder, a weld, a crimp, another appropriate technique, or a combination thereof.

First conductor 148 and second conductor 150 may be configured to maintain electrical connection with conductive fillers 125 of inner conductor 122 while inner lead 114 translates relative to outer lead 112 in accordance with techniques of this disclosure. For example, connector 110 may translate as inner lead 114 translates relative to outer lead 112 such that connector 110 remains substantially stationary relative to inner lead 114.

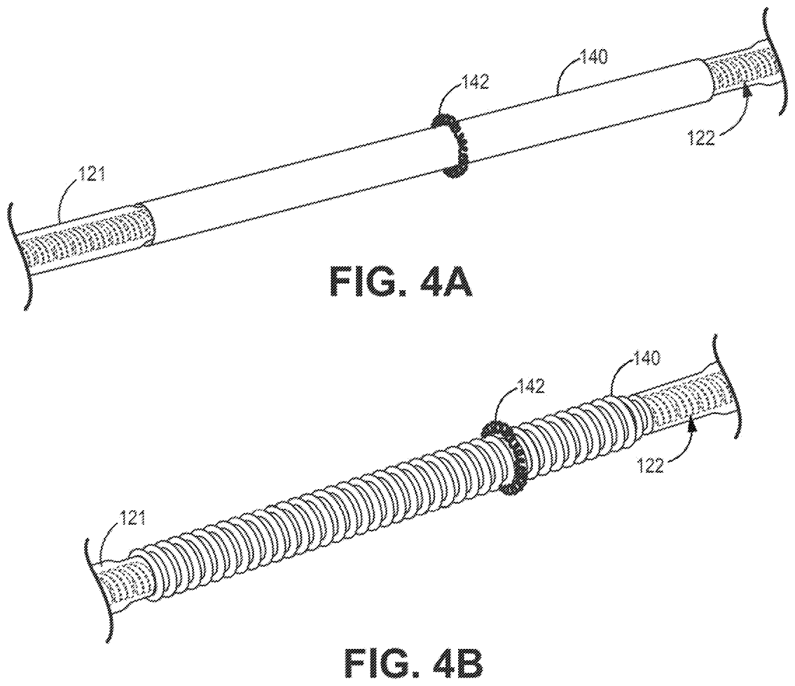

FIGS. 4A and 4B are conceptual diagrams illustrating example configurations of electrical contact 140 surrounding inner lead 114. As shown in FIG. 4A, electrical contact 140 may be formed as a tube defining a passage in which inner lead 114 is positioned. In some examples and as shown in FIG. 4B, electrical contact 140 may be formed as a helical coil. Although electrical contact 140 is not shown in the context of outer lead body 117 for purposes of illustration (e.g., to better expose components surrounded by electrical contact 140), it should be understood that electrical contact 140 may be configured to remain within lumen 138 of inner lead body 117 such that inner lead body 117 protects electrical contact 140. For example, electrical contact 140 may be dimensioned such that even when inner lead 114 translates relative to outer lead 112, the entire length of electrical contact 140 remains within lumen 138.

Electrical contact 140 is configured to electrically communicate with conductive element 142 positioned between outer lead 112 and inner lead 114. Electrical contact 140 may be electrically insulated from inner conductor 122 (e.g., by inner lead body 121). As described above, electrical contact 140 may be electrically connected to one or more of conductive fillers 125, such as conductive filler 125B. Electrical contact 140 may be electrically connected to conductive element 142 (e.g., a coiled ring compressing electrical contact 140), which in turn may be electrically connected to outer conductor 130. In this way, connector 110 may energize outer lead electrode 116 via electrical contact 140 and conductive element 142.

As shown in FIGS. 4A and 4B, inner lead body 121 may mechanically support conductive element 142. Conductive element 142 may translate relative to outer lead 112 when inner lead 114 translates relative to outer lead 112. In examples, conductive element 142 is a coiled spring formed as a ring that is configured to surround electrical contact 140 and mechanically communicate with at least a portion of an outer perimeter of electrical contact 140.

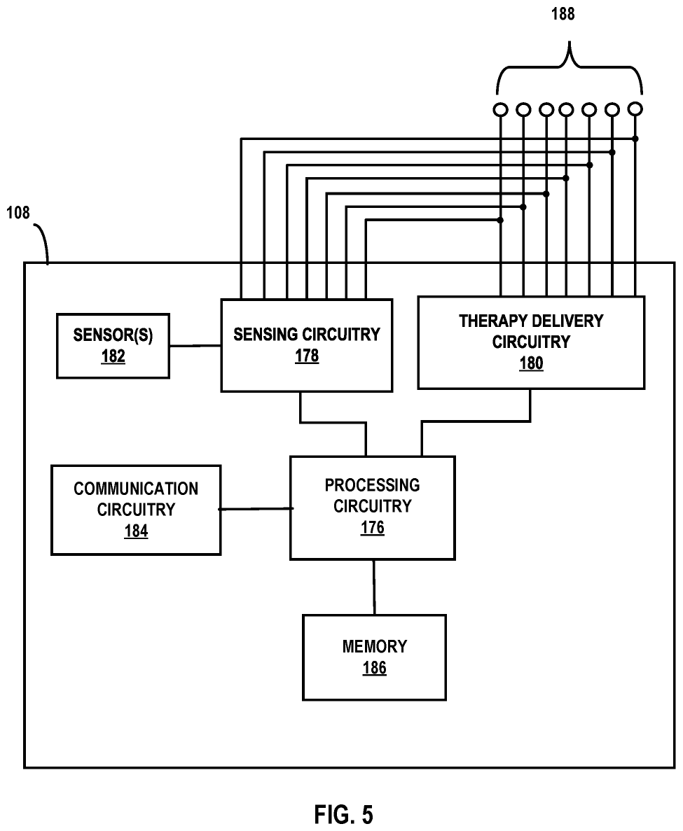

FIG. 5 is a functional block diagram illustrating an example configuration of medical device 111. As shown in FIG. 5, medical device 111 includes processing circuitry 176, sensing circuitry 178, therapy delivery circuitry 180, sensors 182, communication circuitry 184, and memory 186. In some examples, memory 186 includes computer-readable instructions that, when executed by processing circuitry 176, cause medical device 111 and processing circuitry 176 to perform various functions attributed to medical device 111 and processing circuitry 176 herein. Memory 186 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processing circuitry 176 may include fixed function circuitry and/or programmable processing circuitry. Processing circuitry 176 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 176 may include multiple components, such as any combination of one or more microprocessors, one or more controllers, one or more DSPs, one or more ASICs, or one or more FPGAs, as well as other discrete or integrated logic circuitry. The functions attributed to processing circuitry 176 herein may be embodied as software, firmware, hardware or any combination thereof.

In some examples, processing circuitry 176 may receive (e.g., from an external device), via communication circuitry 184, a respective value for each of a plurality of cardiac sensing parameters, cardiac therapy parameters (e.g., cardiac pacing parameters), and/or electrode vectors. Processing circuitry 176 may store such parameters and/or electrode vectors in memory 186.

Therapy delivery circuitry 180 and sensing circuitry 178 are electrically coupled to electrodes 188, which may correspond to outer lead electrode 116, inner lead electrode 118, and/or other electrodes of implantable medical lead 102, via connector 110. Processing circuitry 176 is configured to control therapy delivery circuitry 180 to generate and deliver electrical therapy to heart 108 via electrodes 188. Electrical therapy may include, for example, pacing pulses, or any other suitable electrical stimulation. Processing circuitry 176 may control therapy delivery circuitry 180 to deliver electrical stimulation therapy via electrodes 188 according to one or more therapy parameter values, which may be stored in memory 186. Therapy delivery circuitry 180 may include capacitors, current sources, and/or regulators, in some examples.

In general, electrodes 188 of medical device 111 may be ring electrodes, multi-polar electrodes (e.g., segmented electrodes), or any other type of electrode configured to sense electrical signals from and deliver therapy signals to heart 108. Each of electrode 188 may be configured to independently deliver simulation therapy, which may facilitate correctly placing electrodes 188 and/or obtaining a better electrical signal (lower threshold, lower impedance, etc.).

In addition, processing circuitry 176 is configured to control sensing circuitry 178 to monitor signals from electrodes 188 in order to monitor electrical activity of heart 108. Sensing circuitry 178 may include circuits that acquire electrical signals, such as filters, amplifiers, and analog-to-digital circuitry. Electrical signals acquired by sensing circuitry 178 may include intrinsic and/or paced cardiac electrical activity, such as atrial depolarizations and/or ventricular depolarizations. Sensing circuitry 178 may filter, amplify, and digitize the acquired electrical signals to generate raw digital data. Processing circuitry 176 may receive the digitized data generated by sensing circuitry 178. In some examples, processing circuitry 176 may perform various digital signal processing operations on the raw data, such as digital filtering. In some examples, in addition to sensing circuitry 178, medical device 111 optionally may include sensors 182, which may be one or more pressure sensors and/or one or more accelerometers, as examples. Communication circuitry 184 may include any suitable hardware (e.g., an antenna), firmware, software, or any combination thereof for communicating with another device, e.g., external to the patient.

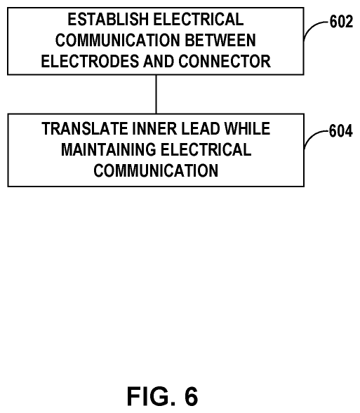

A technique for operating medical device system 100 is illustrated in FIG. 6. As shown in FIG. 6, connector 110 may establish electrical communication between processing circuitry 176 and electrodes 188 of outer lead 112 and inner lead 114 (602). Connector 110 may be a quadripolar connector or IS-4 type connector and may be configured such that processing circuitry 176 can electrically communicate with outer lead 112 and inner lead 114 substantially independently. For example, processing circuitry 176 may transmit or sense a first signal via outer lead 112 and transmit or sense a second signal via inner lead 114. In examples, connector 110 includes at least first conductor 148 that establishes electrical communication between inner lead 114 and processing circuitry 176 and second conductor 150 that establishes electrical communication between outer lead 112 and processing circuitry 176. In examples, connector 110 includes connector body 147 mechanically supporting first conductor 148 and second conductor 150. Connector body 147 may maintain first conductor 148 and second conductor 150 substantially stationary relative to each other. Connector body 147 may electrically insulate at least some portion of first conductor 148 from second conductor 150, and vice-versa.

Connector 110 may energize, via first conductor 148 and second conductor 150 (and other conductors of connector 110), outer lead electrode 116 and inner lead electrode 118 (and other electrodes of implantable medical lead 102) by delivering electrical stimulation from therapy delivery circuitry 180 of medical device 111. First conductor 148 may electrically connect with conductive filler 125A of inner conductor, and second conductor 150 may electrically connect with conductive filler 125B. First conductor 148 and second conductor 150 may maintain electrical connection with inner conductor 122 while inner lead 114 translates relative to outer lead 112.

As further shown in FIG. 6, inner lead 114 may translate relative to outer lead 112 (604). In examples, inner lead 114 is configured to receive a force that causes inner lead 114 to translate relative to outer lead 112. Inner lead 114 and/or outer lead 112 may be configured such that a physician may control a depth to which inner lead 114 penetrates the tissue (e.g., ventricular tissues), allowing inner lead 114 to be substantially positioned at a predetermined location based on, for example, pace mapping. In some examples, inner lead 114 may translate relative to outer lead 112 when inner lead 114 rotates relative to inner lead 112.

Inner lead 114 may be electrically connected to connector 110 to enable electrical communication as inner lead 114 translates relative to outer lead 112. Electrical contact 140 and conductive element 142 may enable inner lead 114 to maintain electrical communication with connector 110 while inner lead 114 translates relative to outer lead 112. In this way, both outer lead 112 and inner lead 114 may electrically communicate with a single connector (e.g., connector 110), and inner lead 114 may translate (e.g., slide) relative to outer lead 112 while outer lead 112 and inner lead 114 maintain electrical communication with connector 110.

The techniques described in this disclosure may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices.

In one or more examples, the functions described in this disclosure may be implemented in hardware, software, firmware, or any combination thereof. If implemented in software, the functions may be stored, as one or more instructions or code, on a computer-readable medium and executed by a hardware-based processing unit. Computer-readable media may include computer-readable storage media forming a tangible, non-transitory medium. Instructions may be executed by one or more processors, such as one or more DSPs, ASICs, FPGAs, general purpose microprocessors, or other equivalent integrated or discrete logic circuitry. Accordingly, the term “processor,” as used herein may refer to one or more of any of the foregoing structures or any other structure suitable for implementation of the techniques described herein.

In addition, in some respects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques may be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Various examples of the disclosure have been described. Any combination of the described systems, operations, or functions is contemplated. These and other examples are within the scope of the following claims.

What is claimed is:

1. An implantable medical lead comprising:
a connector configured to electrically communicate with processing circuitry of a medical device;
an outer lead comprising:
an outer lead electrode; and
an outer conductor electrically connected to the outer lead electrode;
an inner lead positioned within a lumen defined by the outer lead, wherein the inner lead is configured to translate within the lumen relative to the outer lead, and wherein the inner lead comprises:
an inner lead electrode; and
an inner conductor electrically connected to the connector, wherein the inner conductor is configured to establish electrical communication between the inner lead electrode and the connector, and wherein the connector is configured to establish electrical communication between the inner lead electrode and the processing circuitry using the inner conductor;
an electrical contact electrically connected to the outer conductor; and
a conductive element positioned within the lumen,
wherein the conductive element is electrically connected to the connector,
wherein the conductive element is configured to establish electrical communication between the connector and the electrical contact as the inner lead translates relative to the outer lead, and
wherein the connector is configured to establish electrical communication between the outer lead electrode and the processing circuitry using the outer conductor when the conductive element establishes electrical communication between the connector and the electrical contact.

2. The implantable medical lead of claim 1, wherein the inner conductor comprises a first conductive filler and a second conductive filler, wherein the connector comprises a first conductor and a second conductor, wherein the first conductive filler is electrically connected to the first conductor, and wherein the second conductive filler is electrically connected to the second conductor.

3. The implantable medical lead of claim 1, wherein the electrical contact is positioned within the lumen of the outer lead.

4. The implantable medical lead of claim 3, wherein the electrical contact is formed as a tube defining a passage within which the inner lead is positioned.

5. The implantable medical lead of claim 1, wherein the electrical contact is elongate and configured to be flexible.

6. The implantable medical lead of claim 1, wherein the electrical contact is electrically connected to the second conductive filler of the inner conductor.

7. The implantable medical lead of claim 1, wherein the electrical contact is configured to remain substantially stationary relative to the inner lead.

8. The implantable medical lead of claim 1, wherein the conductive element is positioned between the inner lead and the outer lead, and wherein the outer lead mechanically supports the conductive element, and wherein the conductive element remains stationary with respect to the outer lead when the inner lead translates relative to the outer lead.

9. The implantable medical lead of claim 1, wherein the conductive element is positioned between the inner lead and the outer lead, and wherein the inner lead mechanically supports the conductive element, and the conductive element translates relative to the outer lead when the inner lead translates relative to the outer lead.

10. The implantable medical lead of claim 1, wherein the conductive element is positioned between the inner lead and the outer lead, and wherein the conductive element is a coiled spring formed as a ring that is configured to:
surround the inner lead; and
mechanically communicate with at least a portion of an outer perimeter of the inner lead.

11. The implantable medical lead of claim 1, wherein the outer conductor comprises a conductive coil electrically connected to the outer lead electrode.

12. The implantable medical lead of claim 1, wherein the outer conductor defines a helical coil surrounding the inner lead.

13. The implantable medical lead of claim 1, wherein the outer lead includes an outer lead body mechanically supporting the outer conductor, wherein the outer lead body defines the lumen.

14. The implantable medical lead of claim 13, wherein the outer conductor is embedded in the outer lead body.

15. The implantable medical lead of claim 1, further comprising a fixation mechanism configured to secure the outer lead to tissues of a patient.

16. The implantable medical lead of claim 15, wherein the fixation mechanism mechanically supports the outer lead electrode.

17. The implantable medical lead of claim 15, wherein the fixation mechanism comprises a helix or a plurality of tines.

18. The implantable medical lead of claim 1, wherein the outer lead defines an outer lead axis, and wherein the inner lead is configured to translate substantially parallel to the outer lead axis.

19. The implantable medical lead of claim 1, further comprising:
a proximal seal, proximate to a proximal end of the outer lead and disposed within the lumen of the outer lead, configured to prevent fluid from migrating into the outer lead through the proximal end of the outer lead; and
a transition ring, positioned distal to the proximal seal and disposed within the lumen of the outer lead, configured to control translation of the inner lead relative to the outer lead by contacting a support component configured to house at least a portion of the conductive element.

20. The implantable medical lead of claim 1, wherein the inner lead supports one or more electrodes in addition to the inner lead electrode.

21. The implantable medical lead of claim 20, wherein the one or more electrodes in addition to the inner lead electrode are positioned near a distal end of the inner lead.

22. A system comprising:
an implantable medical lead comprising:
a connector configured to electrically communicate with processing circuitry of a medical device;
an outer lead comprising:
an outer lead electrode; and
an outer conductor electrically connected to the outer lead electrode;
an inner lead positioned within a lumen defined by the outer lead, wherein the inner lead is configured to translate within the lumen relative to the outer lead, and wherein the inner lead comprises:
an inner lead electrode; and
an inner conductor electrically connected to the connector, wherein the inner conductor is configured to establish electrical communication between the inner lead electrode and the connector, and wherein the connector is configured to establish electrical communication between the inner lead electrode and the processing circuitry using the inner conductor;
an electrical contact electrically connected to the outer conductor; and
a conductive element positioned within the lumen,
wherein the conductive element is electrically connected to the connector,
wherein the conductive element is configured to establish electrical communication between the connector and the electrical contact as the inner lead translates relative to the outer lead, and
wherein the connector is configured to establish electrical communication between the outer lead electrode and the processing circuitry using the outer conductor when the conductive element establishes electrical communication between the connector and the electrical contact; and
an implantable medical device configured to electrically couple to the implantable medical lead, the implantable medical device comprising processing circuitry configured to deliver therapy signals to a heart of a patient using at least one of the inner lead electrode or the outer lead electrode.

23. The system of claim 22, wherein the processing circuitry is configured to sense cardiac signals from a heart of a patient using at least one of the inner lead electrode or the outer lead electrode.

24. The system of claim 22, wherein the processing circuitry is configured to at least one of deliver therapy signals to or sense cardiac signals from ventricular tissue of a heart using at least one of the inner lead electrode or the outer lead electrode.

25. The system of claim 22, wherein the processing circuitry is configured to deliver therapy signals to a conduction system of a heart using at least one of the inner lead electrode or the outer lead electrode.

26. A method comprising:
translating an inner lead of an implantable medical lead comprising a connector configured to electrically communicate with processing circuitry of a medical device, wherein the inner lead is positioned within a lumen defined by an outer lead of the implantable medical lead, and wherein the outer lead comprises:
an outer lead electrode; and
an outer conductor electrically connected to the outer lead electrode; and
wherein the inner lead comprises:
an inner lead electrode; and
an inner conductor electrically connected to the connector, wherein the inner conductor is configured to establish electrical communication between the inner lead electrode and the connector, and wherein the connector is configured to establish electrical communication between the inner lead electrode and the processing circuitry using the inner conductor; and
establishing electrical communication between the outer conductor and the connector, wherein an electrical contact and a conductive element maintain the electrical communication between the outer conductor and the connector as the inner lead translates relative to the outer lead, wherein the electrical contact is electrically connected to the outer conductor, wherein the conductive element is positioned within the lumen defined by the outer lead, wherein the conductive element is electrically connected to the connector, wherein the conductive element is configured to establish electrical communication between the connector and the electrical contact as the inner lead translates relative to the outer lead, and wherein the connector is configured to establish electrical communication between the outer lead electrode and the processing circuitry using the outer conductor when the conductive element establishes electrical communication between the connector and the electrical contact.
